(19) United States
(12) Patent Application Publication
(10) Pub. No.: US 2008/0287989 A1
(43) Pub. Date: Nov. 20, 2008

(54) Tissue holding implants

(75) Inventors: Thomas Weisel, Ventura, CA (US); Roger Pisarnwongs, Valencia, CA (US)

Correspondence Address: KOPPEL, PATRICK & HEYBL, 555 ST. CHARLES DRIVE, SUITE 107, THOUSAND OAKS, CA, 91360, US

(73) Assignee: ARCH DAY DESIGN, LLC

(21) Appl. No.: 12/152,361

(22) Filed: May 13, 2008

Related U.S. Application Data
(60) Provisional application No. 60/930,713, filed on May 17, 2007.

(51) Int. Cl. A61B 17/08

(52) U.S. Cl. 606/220

(57) ABSTRACT

Tissue holding implants which enable two or more layers of tissue to be held in approximation for an extended period. One and two-piece implants are described, made from materials that are biocompatible, and may also be biodegradable. Some embodiments include a portion which is deformed to capture the tissues to be held, and others are made from a super-elastic or shape memory material and hold the tissues in approximation when the implant is reverted to a known preformed shape.

### RELATED APPLICATIONS

This application claims the benefit of provisional patent application No. 60/930,713 to Thomas Weisel and Roger Pisarnwongs, filed May 17, 2007.

### BACKGROUND OF THE INVENTION

1. Field of the Invention

This invention relates generally to tissue holding devices, and more particularly to implants for holding two or more tissues in approximation.

2. Description of the Related Art

During medical procedures such as surgery, it is often necessary to join two or more tissues in approximation until healing has occurred. It is generally important that the doctor be able to perform this task safely and quickly. However, for some procedures, conventional methods of joining tissues can be unsatisfactory.

Septoplastic surgery is an example of such a procedure. During a typical septoplastic procedure, the surgeon will peel the mucosa from each side of the septal cartilage, modify the cartilage as required, and then reattach the mucosa. This is often done with a suture being passed back and forth through the 2 or 3 layers of tissue (mucosa-septum-mucosa or mucosa-mucosa), working alternately through each nostril. This suturing task can sometimes be tedious and time consuming due to swollen tissue and difficult access.

Many other tissue-connecting devices and techniques are known beyond the standard suture. However, for an application such as that described above—i.e., reapproximating mucosa, septum and mucosa after a septoplasty—the only commonly-known connection scheme which places force on the outer mucosa surfaces is the suture, despite the disadvantages noted above.

### SUMMARY OF THE INVENTION

The present invention is directed to tissue holding implants which overcome the problems noted above, in that the described devices are easily and quickly inserted, and enable tissues to be held in approximation for an extended period.

The presented devices are one or two piece implants, made from materials that are biocompatible, and may also be biodegradable. The implants can be used for any tissues in the body that require approximation for an extended period. They can be used in conjunction with many different types of procedures, including septoplastic surgery, wound closure, meniscal repair, shoulder capsulorrhaphy and various laparoscopic procedures. The implants described herein can be used to hold two or more layers of tissue in approximation.

Various implant configurations are described, including implants which include a portion which is deformed to capture the tissues to be held, and others which are made from a super-elastic or shape memory material which hold the tissues in approximation when the implant is reverted to a known preformed shape.

These and other features, aspects, and advantages of the present invention will become better understood with reference to the following description and claims.

### BRIEF DESCRIPTION OF THE DRAWINGS

FIGS. 1-3 are sectional views illustrating one possible embodiment of an implant in accordance with the present invention.

FIGS. 4-5 are sectional views illustrating another possible embodiment of an implant in accordance with the present invention.

FIGS. 6-8 are sectional views illustrating another possible embodiment of an implant in accordance with the present invention.

FIGS. 9-12 FIGs. are sectional views illustrating another possible embodiment of an implant in accordance with the present invention.

FIGS. 13-15 are sectional views illustrating another possible embodiment of an implant in accordance with the present invention.

FIGS. 16-22 are sectional views illustrating another possible embodiment of an implant in accordance with the present invention.

FIGS. 23-27 are sectional views illustrating another possible embodiment of an implant in accordance with the present invention.

FIGS. 28-32 are sectional views illustrating another possible embodiment of an implant in accordance with the present invention.

FIGS. 33-39 are sectional views illustrating another possible embodiment of an implant in accordance with the present invention.

FIGS. 40-44 are sectional views illustrating another possible embodiment of an implant in accordance with the present invention.

FIGS. 45-46 are sectional views illustrating another possible embodiment of an implant in accordance with the present invention.

FIGS. 47-49 are sectional views illustrating another possible embodiment of an implant in accordance with the present invention.

FIGS. 50-52 are sectional views illustrating another possible embodiment of an implant in accordance with the present invention.

FIGS. 53-55 are sectional views illustrating another possible embodiment of an implant in accordance with the present invention.

FIGS. 56-57 are sectional views illustrating another possible embodiment of an implant in accordance with the present invention.

### DETAILED DESCRIPTION OF THE INVENTION

The present tissue holding devices are one or two-piece implants, made from materials that are biocompatible, and may also be biodegradable. They can be used in conjunction with many different types of procedures, including septoplastic surgery, wound closure, meniscal repair, shoulder capsulorrhaphy and various laparoscopic procedures. The following text will use septoplastic surgery as an exemplary application for the implants, but it should be noted that these devices can be used for any tissues in the body that require approximation for an extended period. Also, though the figures accompanying the examples described below show two pieces of tissue, the present implants can be used to hold more than two tissues, which may be of different types and have different dimensions. Note that the use of block arrows in the figures indicate movement or force in the direction indicated.

One embodiment of an implant for holding two or more tissues in approximation generally comprises a head portion and a shaft which extends from the head portion, with at least a portion of the implant being deformable. To use, the shaft is inserted through the tissues to be held and the deformable portion is deformed. The implant is designed to hold the tissues in approximation when its deformable portion is deformed.

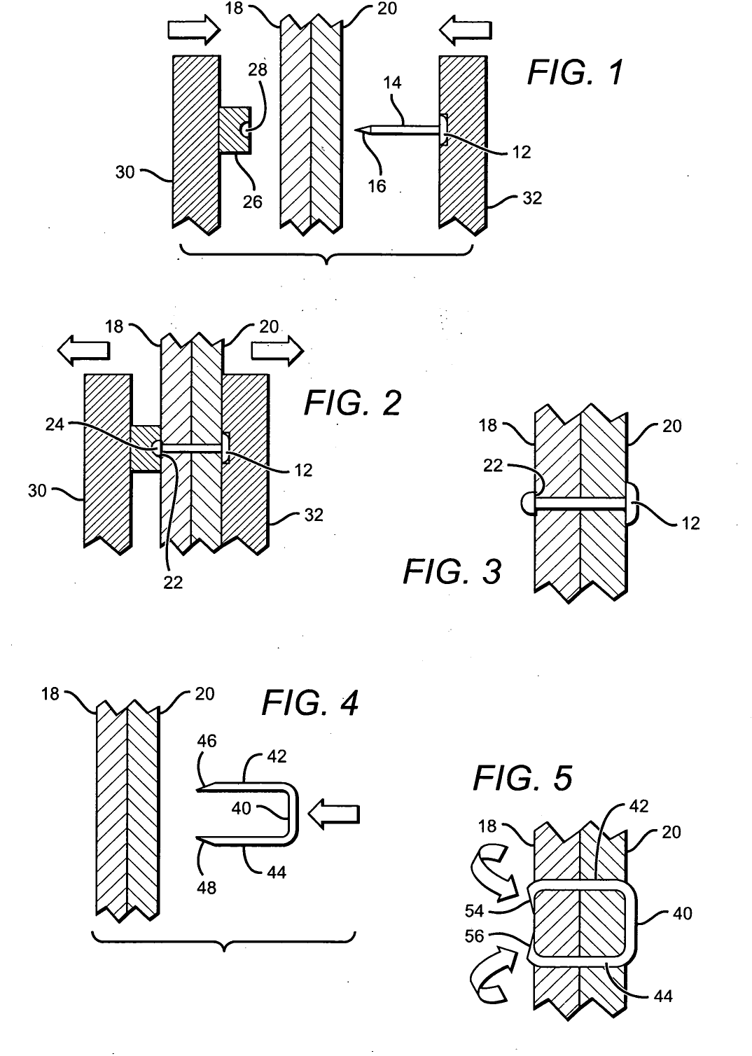

An exemplary embodiment is shown in FIGS. 1-3. The implant comprises a head portion 12 and a shaft 14 which extends from the head portion. The end of the shaft opposite the head portion comes to a point 16, to aid in the piercing of tissue. In this embodiment, point 16 is deformable.

In use (see FIG. 2), shaft 14 is inserted through the tissues (18, 20) to be held, and point 16 is then deformed. This results in head portion 12 pressing against tissue 20, and the tissue-side surface 22 of deformed point 24 pressing against tissue 18, so that tissues 18 and 20 are held in approximation between head portion 12 and the tissue-side surface 22 (see FIG. 3).

A deforming die 26 can be provided to deform point 16. In this example, a pocket 28 in die 26 is brought into contact with point 16, causing it to deform into a shape (24) that enables the tissues to be held. The deforming die can be arranged to deform point 16 is a variety of ways, such as heat or mechanical vibration. Blocks 30 and 32 may be used to bring die 26 into contact with point 16.

Implants in accordance with the present invention are made with materials that are biocompatible, and may also be biodegradable. For example, an implant may comprise an absorbable or non-absorbable biocompatible plastic. Alternatively, an implant may comprise a biocompatible metal, such as stainless steel.

Some embodiments require or are benefited by the use of a super-elastic or shape memory plastic or metal. For example, an implant made from a nickel-titanium alloy such as NITINOL can have the characteristic of super-elasticity. Some implant embodiments might alternatively be made from a material which exhibits a shape memory behavior; some NITINOL alloys possess this property. Implants made from a super-elastic or shape memory material are capable of being formed into an initial or ‘compressed’ shape suitable for insertion into the tissues to be held, and then reverting to a known ‘preformed’ shape. A super-elastic material reverts to its preformed shape when unconstrained, while a shape memory material reverts to its preformed shape in response to an associated activation means, such as heat. For instance, if the activation temperature of the shape memory material is below 37° C., the implant will revert to its preformed state when subjected to normal body temperatures. Note that an implant which is at least partly made from a super-elastic or shape memory material is referred to herein as ‘deformable’.

Another possible embodiment is shown in FIGS. 4 and 5. Here, a head portion 40 has first and second ends, from which first and second shafts 42, 44 extend. The ends 46, 48 of the shafts opposite head portion 40 are pointed and deformable, such that the shafts and head portion form a staple. In use, the staple pierces the tissues to be held (18, 20) with points 46, 48, which are then deformed such that the tissues are held between deformed points 54, 56 and the staple's head portion 40, as shown in FIG. 5.

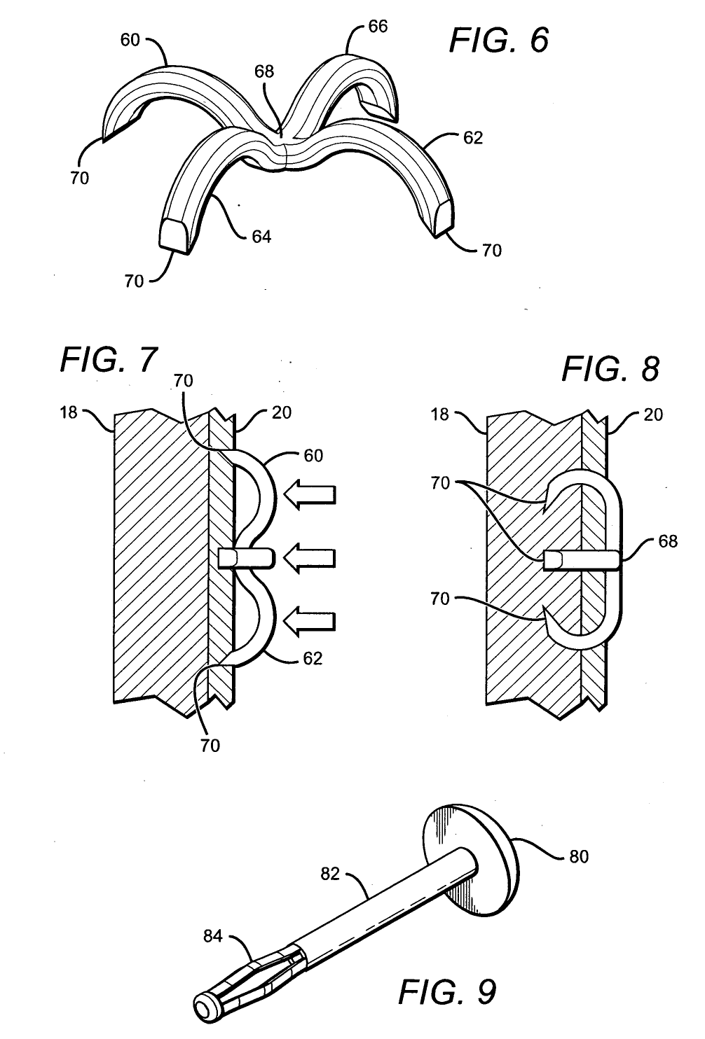

FIGS. 6-8 depict another embodiment which resembles a staple. Here, however, there are three or more shafts 60, 62, 64, 66 extending from the implant's head portion 68. The ends of all the shafts (70) have leading points and are deformable, the shafts and head portion thereby forming a multi-legged staple.

When force is applied to the legs as shown in FIG. 7, the staple deforms as shown in FIG. 8, with points 70 piercing the tissues 18, 20 and curling inward to capture the various tissue layers. Thus, when shafts 60, 62, 64 and 66 are inserted through the tissues to be held and their deformable points are deformed, the tissues are held between head portion 68 and deformed portions 70.

Alternatively, the implant shown in FIGS. 6-8 could be made from a super-elastic or shape memory material. In this case, the implant's compressed shape would be as shown in FIG. 7, and its preformed shape would be as shown in FIG. 8. If the implant is made from an appropriate temperature-sensitive shape memory material, forcing the implant into the tissue and thereby exposing it to a higher temperature causes it to revert to its preformed state. For these embodiments, points 70 would not have to be deformable.

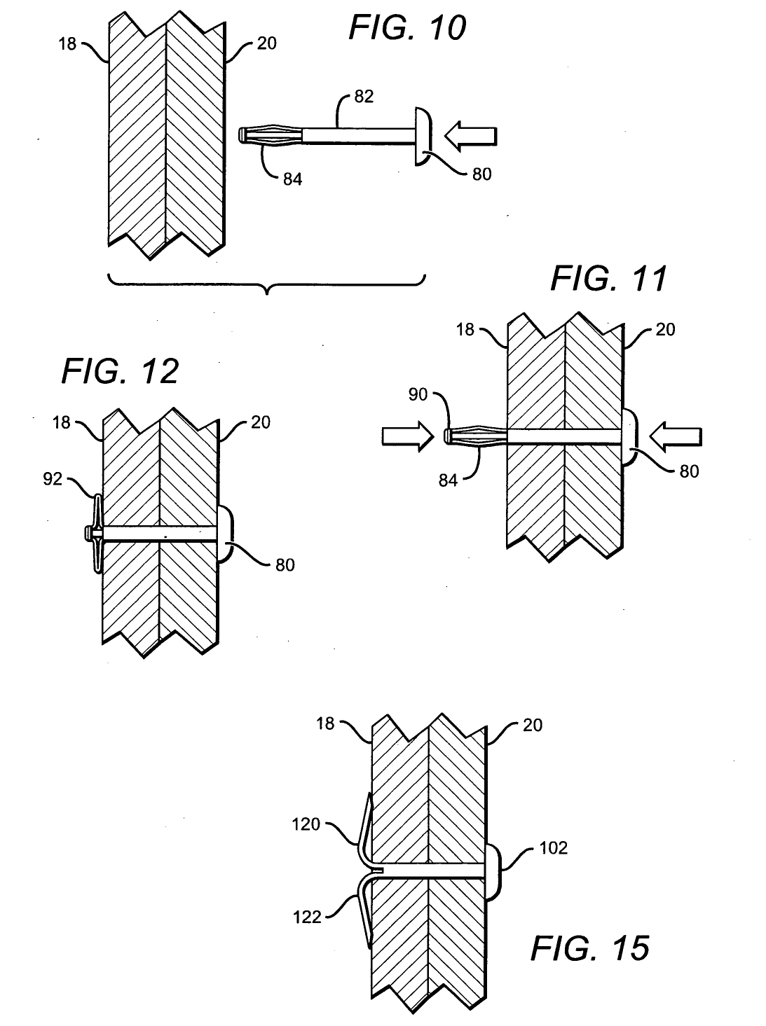

Another possible embodiment is illustrated in FIGS. 9-12. This implant comprises a head portion 80 and a shaft 82 which terminates at one or more molly bolt-like collapsible legs 84. As shown in FIGS. 10 and 11, the implant is pushed through tissues to be held 18, 20. Rams (not shown) force head portion 80 and the ends 90 of collapsible legs 84 towards each other, such that the legs are forced to collapse (92); this is shown in FIG. 12. When legs 84 are collapsed in this way, tissues 18 and 20 are held between head portion 80 and collapsed legs 92.

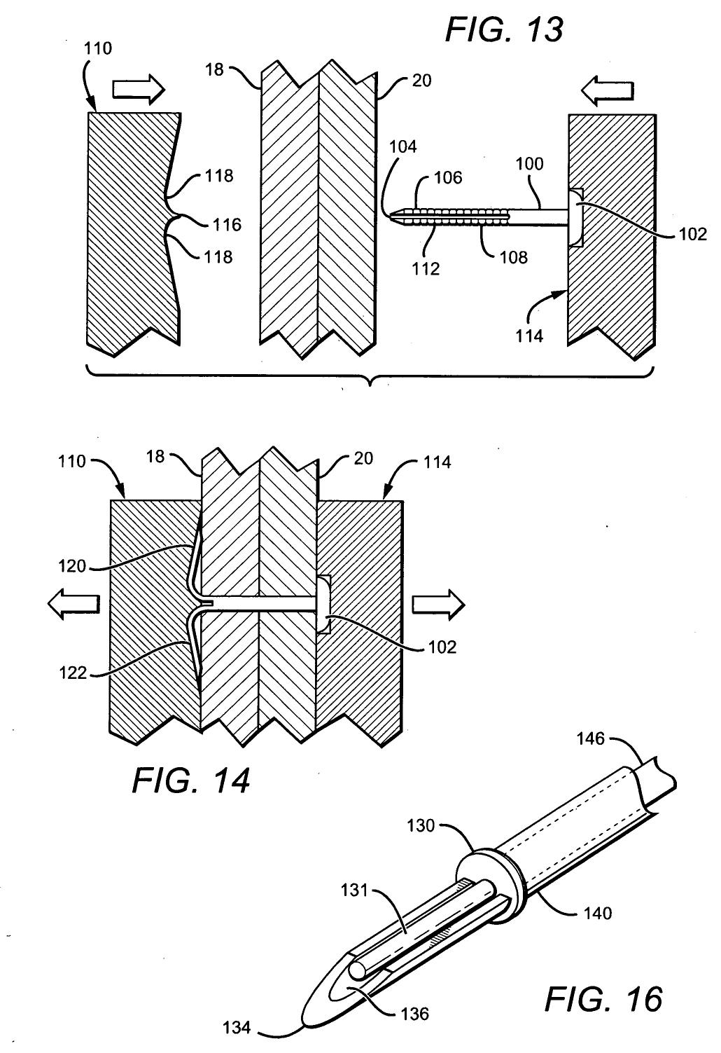

FIGS. 13-15 illustrate another possible embodiment, in which the shaft 100 extending from a head portion 102 contains one or more slits 104, which extend from the tip of the shaft towards the head portion. The portions of the shaft between the slits (106, 108) are deformable. The ends of portions 106, 108 are preferably pointed, to aid in piercing the tissues to be held (18, 20).

A deforming ram 110 is preferably employed to split shaft 100 along its slits 104 when brought into contact with shaft portions 106, 108. Portions 106, 108 may include relief notches 112 that allow the shaft to be more easily deformed after tissue insertion. FIG. 13 shows two rams, 110 and 114, pressing against head portion 102 and shaft portions 106, 108, respectively. Ram 110 has an optional protrusion 116 to help split the shaft along slits 104. Then, each of shaft portions 106, 108 follows one of the forming curves 118 of ram 110 so that the deformed legs 120, 122 press against the outer surface of tissue 18. In this way, tissues 18, 20 are then held between head portion 102 and deformed legs 120, 122.

For some embodiments, insertion of the implant through the tissues is preferably accomplished using a needle into or onto which an implant can be loaded. The implant and needle are designed such that, when made to pierce the tissues to be held, the implant's shaft is delivered through the tissues along with needle. In some applications, the needle and implant are preferably curved; this allows the implant to go in at a plane of tissue, go through the tissue's thickness and into a second piece of tissue, and then curve back out and exit at the original plane.

A ram may be used to push an implant out of a needle in which it is loaded. A typical ram is shaped like a cylinder or a stick with first and second ends. When the needle is loaded with an implant, the ram is positioned so that its first end abuts against the side of the implant's head portion opposite its shaft. Then, force applied to the ram's second end pushes the implant towards the needle's point such that the implant's shaft is delivered through the tissues along with the needle.

The implant embodiments described herein which are intended for insertion into the tissues to be held using a needle may be arranged such that they have a deformable portion which is deformed to hold the tissues. Alternatively, the implants may comprise a super-elastic or shape memory material, with the implant being in its compressed state while loaded in the needle, and then reverting to its preformed state after being inserted into the tissues and having the needle retracted.

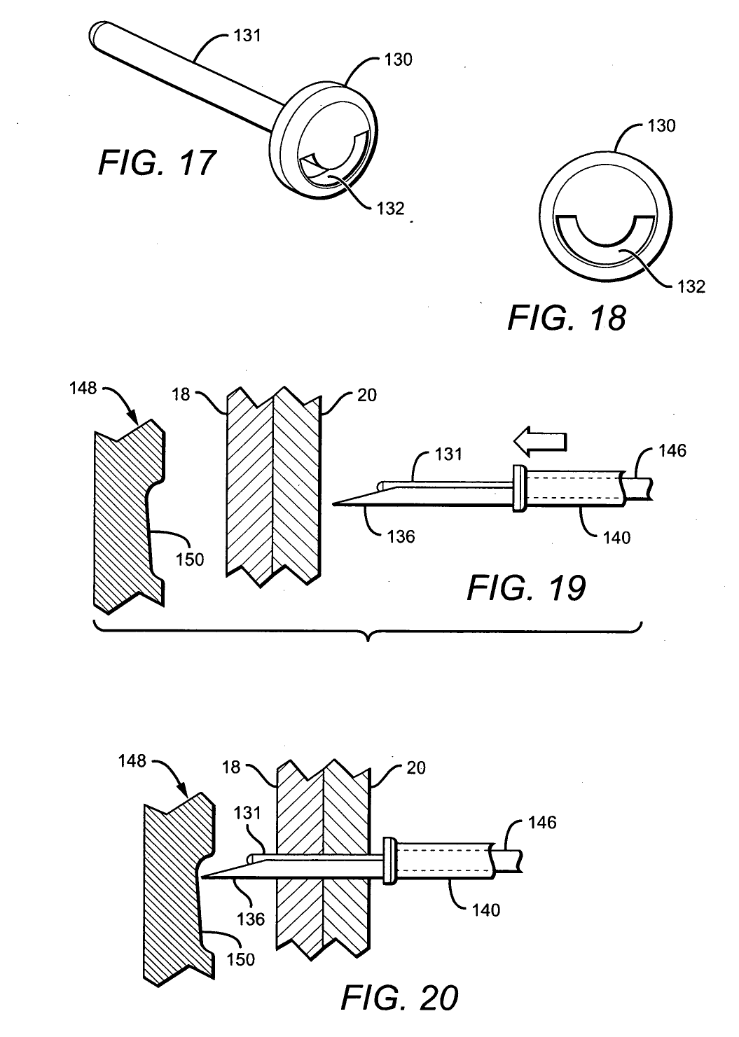

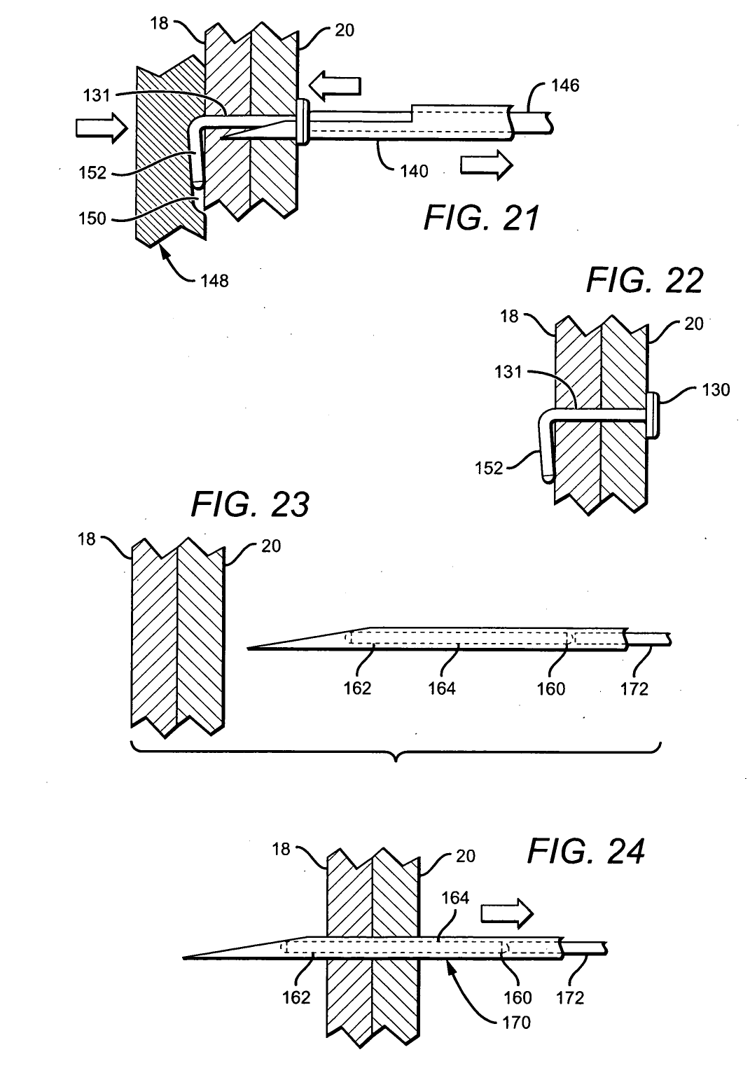

One possible embodiment of a needle-inserted implant is illustrated in FIGS. 16-22. The implant comprises a head portion 130 and a shaft 131, with the head portion including a slot 132 through which the tip 134 and cut portion 136 of a needle extend. Note that the slot can also be open to the outside circumference. The needle also has a base portion 140, which abuts against the side of head portion 130 opposite the shaft when the implant is loaded into the needle. As shown in FIGS. 19 and 20, when the needle pierces the tissues to be held (18, 20), the shaft 131 of the implant is also delivered through the tissues to be held. The implant is preferably pushed from the needle using a cylindrical or stick-shaped ram 146 as described above.

FIGS. 19-21 illustrate the insertion of this implant assuming that the end of shaft 131 is deformable. A forming die 148 having a cavity 150 can be used to push on the end of shaft 131 while the needle is retracted and ram 146 pushes head portion 130 forward, thereby deforming the end 152 of shaft 131. Then, tissues 18, 20 are held between head portion 130 and deformed portion 152.

Note that all or some of shaft 131 could alternatively be made from a super-elastic or shape memory material, which could eliminate the need for a ram 146. As explained above, such materials are capable of being formed into an initial ‘compressed’ shape suitable for insertion into the tissues to be held, and then reverting to a known ‘preformed’ shape when unconstrained (in the case of a super-elastic embodiment), or in response to an associated activation means, such as heat (in the case of a shape-memory embodiment). In this case, the preformed shape would be the bent shaft shown in FIG. 22, and the compressed shape would be the relatively straight shaft required while the implant is loaded in the needle. In use, after the shaft has been delivered through tissues 18, 20, the implant would revert to its preformed shape (with the aid of the associated activation means if made from a shape-memory material) and thereby capture the tissues.

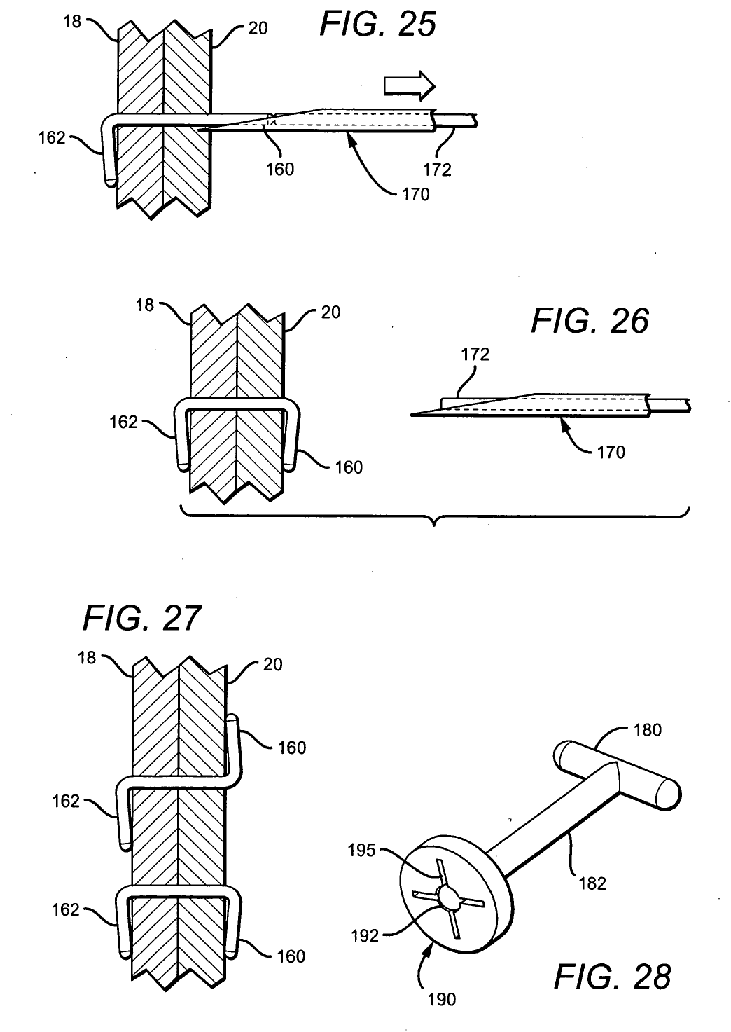

Another possible embodiment of an implant in accordance with the present invention is shown in FIGS. 23-27, in which the head portion 160 and at least a portion 162 of the shaft 164 preferably comprise a shape memory material, though a super-elastic plastic or metal could also be used. This configuration can also be referred to as a preformed wire. The implant is arranged such that the tissues are held in approximation between head portion 160 and the shape memory portion 162 of the shaft when the shaft has been inserted through the tissues and the implant is reverted to its preformed shape.

The implant is delivered into the tissues to be held (18, 20) using a needle 170 and a ram 172, with the implant formed into a compressed shape while loaded in the needle. Needle 170 pierces tissues 18, 20 and is then retracted, with ram 172 pushing the implant from the needle such that it also passes through the tissues. This allows the shape memory portions 160 and 162 to revert to their preformed shapes as shown in FIG. 26, with tissues 18, 20 captured in between. It should be noted that the bends in the implant can be in any plane.

Various shapes could be employed; some examples are shown in FIG. 27. For example, other implants could have a T-shape on one end, and/or multiple radii of curvature along their length to aid in the capture of varying tissue thicknesses. It should also be noted that the implant's cross-section can be round, square, concave, convex, oval, or a variety of other shapes or a combination thereof.

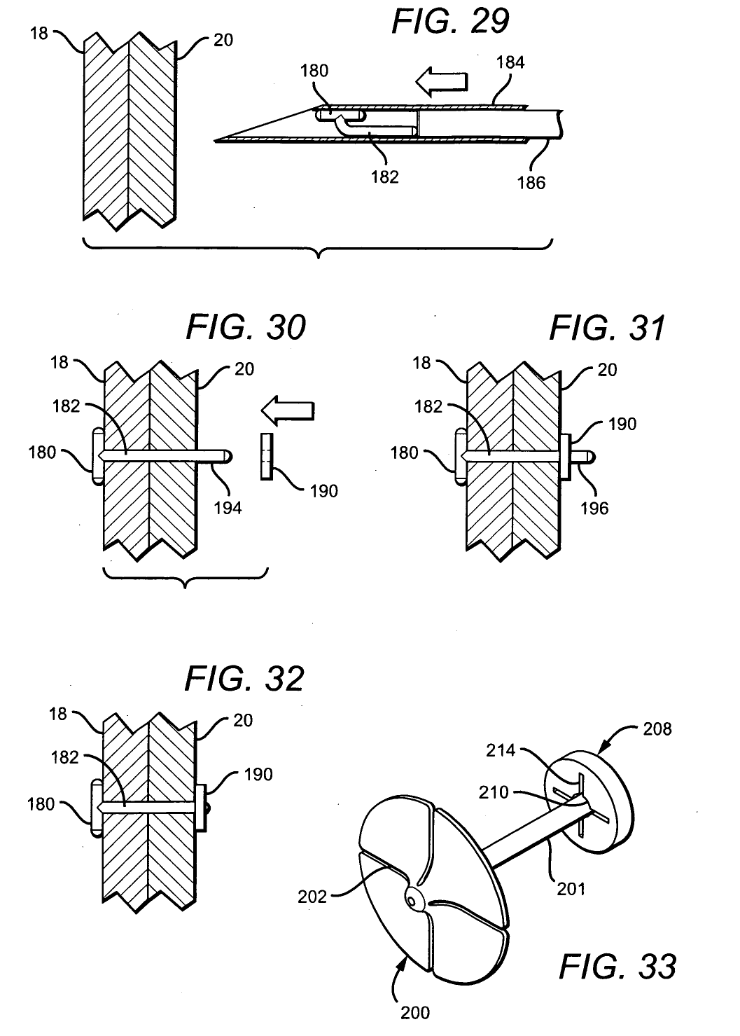

Another possible embodiment is illustrated in FIGS. 28-32. Here, the implant has a T-shape, with a crossbar 180 affixed and perpendicular to one end of the shaft 182. The shaft is inserted through the tissues 18, 20 with the aid of a sharpened needle 184 and a ram 186 as shown in FIG. 29. The implant is made from a super-elastic or shape memory material, so that it can be compressed when loaded in the needle, and then made to revert to its preformed T-shape when shaft 182 has passed through the tissues and the needle is retracted; in this case, shaft 182 is bent and crossbar 180 is loaded parallel to the needle axis. Needle 184 punctures through tissues 18, 20 and ram 186 pushes the implant so that the shaft and crossbar are free to revert to their preformed states on the far side of the tissues.

This embodiment requires a second member 190 which includes an opening 192 for receiving shaft 182, such that the tissues are held in approximation between crossbar 180 and second member 190 when the implant is reverted to its T-shape. As shown in FIGS. 30 and 31, with needle 184 removed, second member 190—here shown as a disk—is placed on the portion of the shaft (194) which has passed completely through the tissues. Disk 190 may optionally include one or more slits 195 to act as a relief for the hole/shaft press fit. The disk and shaft can be held together by many methods, such as a press fit or barbs/ribs on either piece. The portion of the shaft (196) that extends beyond disk 190 is then preferably trimmed, leaving the low profile implant with a large surface area contact on the outer tissue faces, as shown in FIG. 32.

Second member 190 may either be provided with an opening 192 for receiving the shaft, or may be solid until needle 184 punches a hole 192 in the member into which shaft 182 can be pushed. In the former case, the shaft must be aligned with the pre-existing opening; this requirement is eliminated in the latter case.

A similar implant is shown in FIGS. 33-37. Here, the crossbar head portion is replaced by a collapsible umbrella-shaped head portion 200 on shaft 201, with the head portion preferably including slits 202 so it can be collapsed and loaded in a needle 204 for delivery via ram 206 into the tissues to be held 18, 20. The implant is made from a super-elastic or shape memory material, so that it can be compressed when loaded in the needle, and then made to revert to an umbrella shape when shaft 201 has passed through the tissues and the needle is retracted.

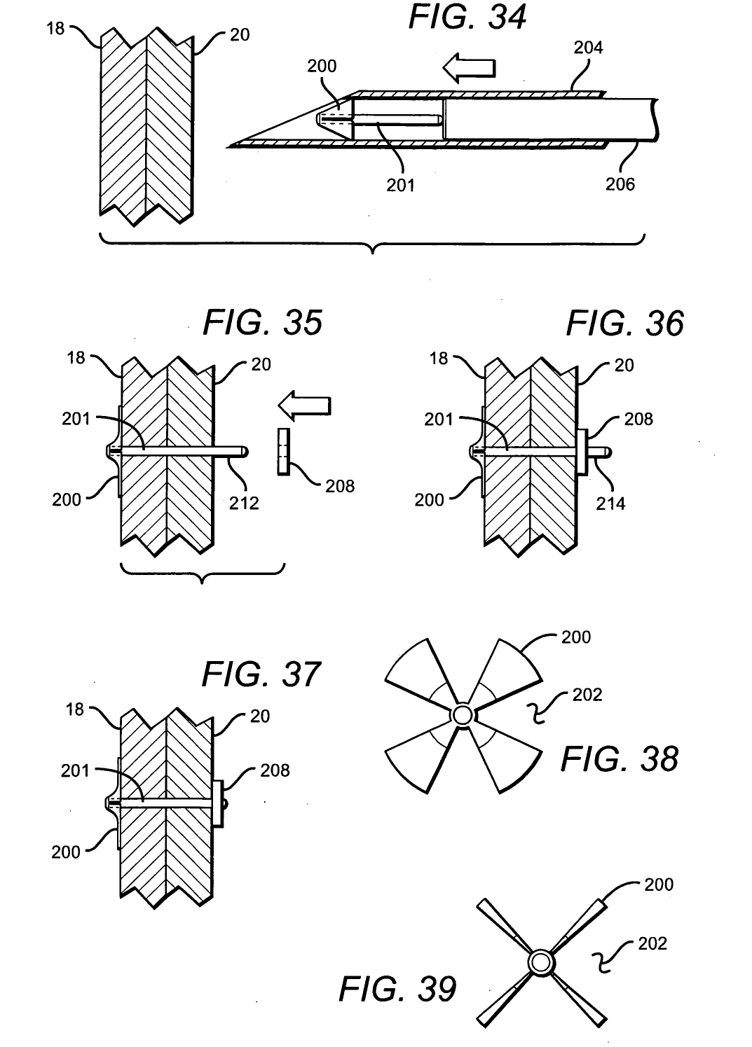

This embodiment also requires a second member 208 which includes an opening 210 for receiving shaft 201, such that the tissues are held in approximation between umbrella-shaped head portion 200 and second member 208 when the head portion is reverted to its umbrella shape. As shown in FIGS. 35-37, with needle 204 removed, second member 208—here shown as a disk—is placed on the portion of the shaft (212) which has passed completely through the tissues. Disk 208 may optionally include one or more slits 214 to act as a relief for the hole/shaft press fit. The portion of the shaft (214) that extends beyond disk 208 is then preferably trimmed. As above, second member 208 may either be provided with an opening 210 for receiving the shaft, or may be solid until needle 204 punches a hole 210 in the member into which shaft 201 can be pushed.

As noted above, the compressible umbrella-shaped head portion preferably includes one or more slits. These act to divide the head portion into two or more segments, each of which can be compressed along the length of the shaft. Note that the gaps between the segments can be wider than those shown in FIG. 33. For example, as shown in the head portion plan views of FIGS. 38 and 39, the slits 202 between the segments of head portion 200 can be made to be generally triangular.

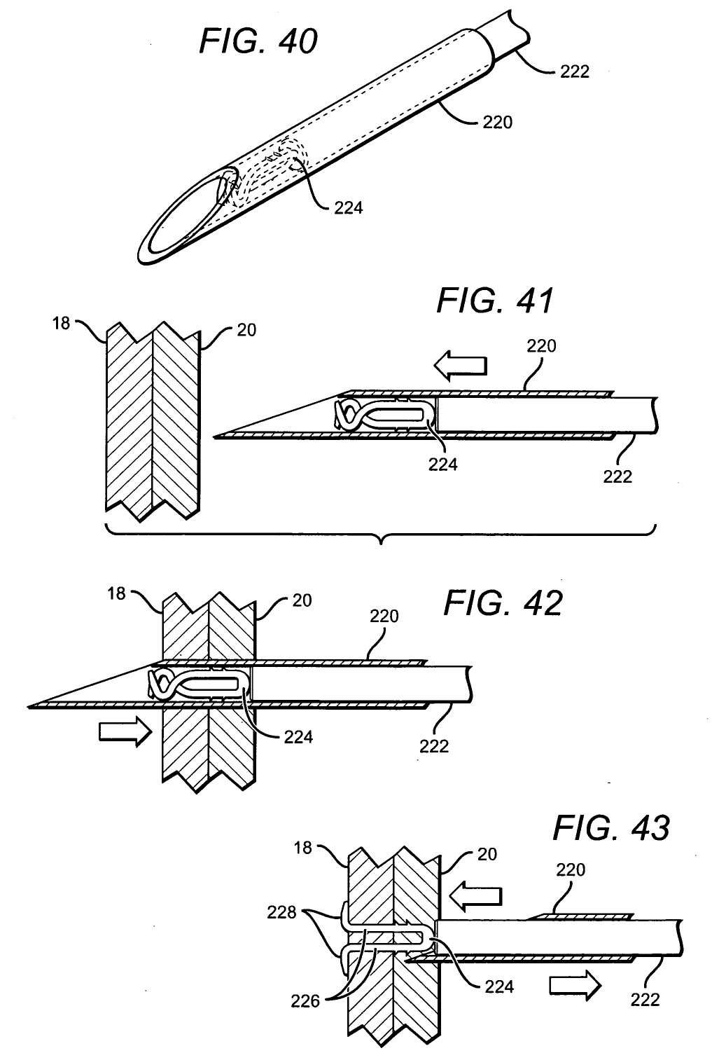

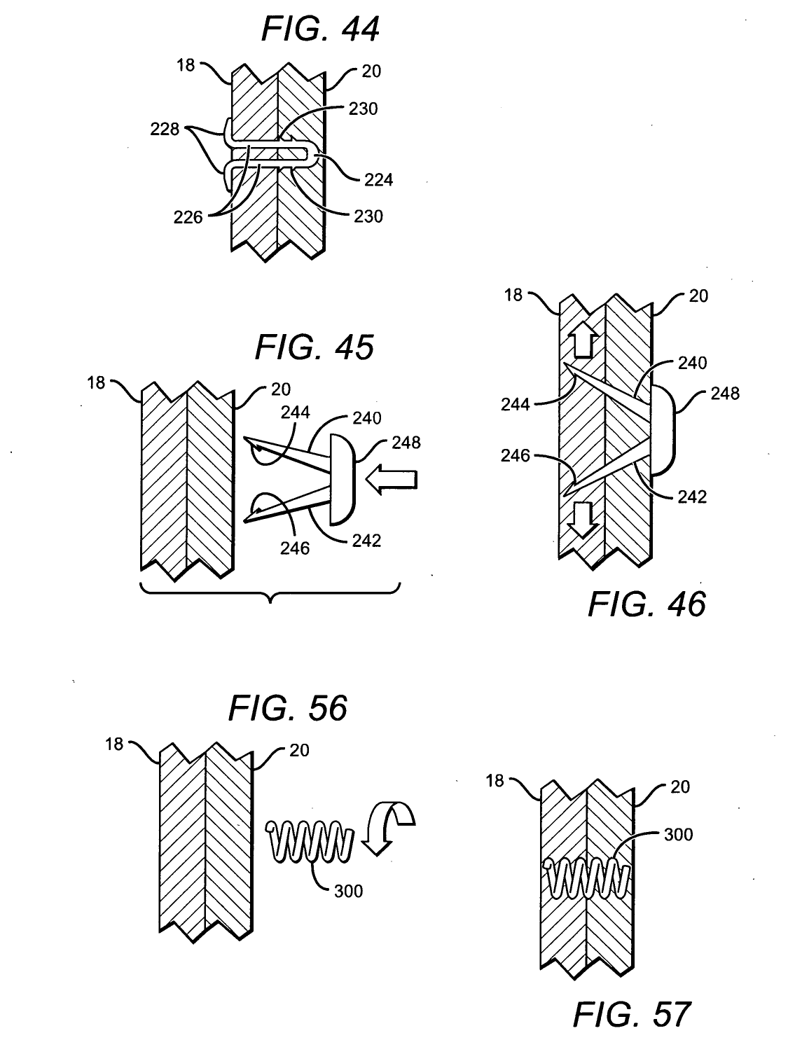

Another implant embodiment for insertion with a needle 220 and ram 222 is shown in FIGS. 40-44. This implant includes two or more shafts which extend from a flexible head portion 224, with each shaft comprising an arm portion 226 coupled to the head portion. A finger portion 228 is on the end of each arm opposite the head portion. The implant is made from a super-elastic or shape memory material, so that it can be compressed when loaded in the needle, and then made to revert to a preformed shape when the needle is retracted. Here, when loaded in needle 220, the implant is a compressed clip. When inserted through tissues 18, 20 and the arms are uncompressed, the tissues are held between fingers 228 and head portion 224, as shown in FIG. 44.

The side of head portion 224 opposite arms 226 may be pointed to aid in piercing the tissues (not shown). Each of arms 226 might also include at least one barb 230 to aid in the fixation of the arms to the tissues.

Another possible approach employs an implant having a head portion and at least one shaft, with each shaft including at least one barb. The barbed shafts are inserted through the tissues, which are held in approximation between the head portion and the barbs. One possible embodiment is illustrated in FIGS. 45-46. This implant includes at least two shafts 240, 242, each of which includes at least one barb 244, 246, is pointed, and extends from head portion 248 at an angle such that, when pushed into the tissues to be held (18, 20), the shafts are spread apart such that an inward force is created that forces the barbs into the tissues.

An alternative embodiment (not shown) would have the shafts extending from the head portion cantered towards each other, with the barbs positioned on the opposite side of the shafts. In this case, when pushed into the tissues to be held, the shafts are pushed together and the barbs are forced into the tissues. An implant having pointed and barbed shafts which extend perpendicularly from the head portion is also contemplated.

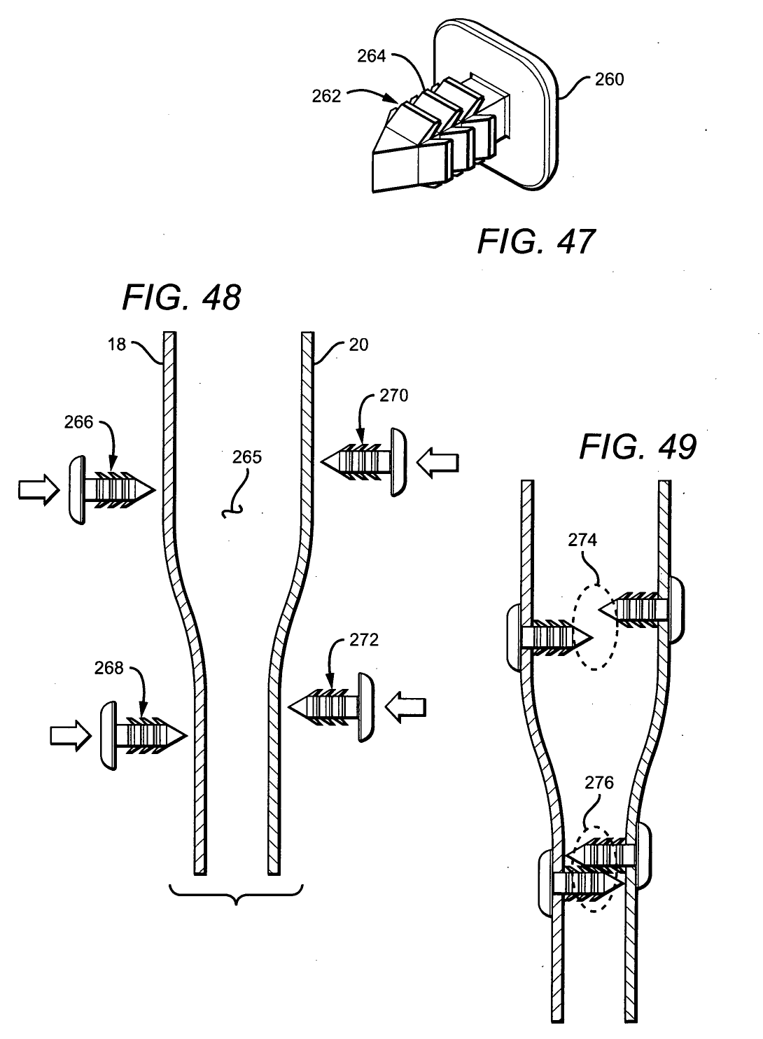

Another implant methodology is presented in FIGS. 47-49. The implant comprises a head portion 260 and at least one shaft 262, with each shaft being pointed and including at least one barb 264. Shaft 262 is shown as having a square-shaped cross-section, but other cross-sectional shapes, such as round or triangular, could also be used. A single implant of this sort could be inserted into the tissues, which would then be held between head portion 260 and barbs 264. Alternatively, two or more implants are used, with at least one implant inserted into the tissues to be gathered 18, 20, 265 from each side; e.g., as shown in FIG. 48, pieces 266 and 268 are inserted into the tissues from the left side, while pieces 270 and 272 are inserted into the tissues from the right side. Tissues 18, 20 are thus held in approximation between each implant's head portion and barbs.

As shown in FIG. 49, implants such as those shown in FIGS. 47 and 48 can be arranged such that two implants inserted into the tissues from opposite directions can be fixated in the tissues by their respective barbs (274), or can intersect and lock onto each other via their respective barbs (276).

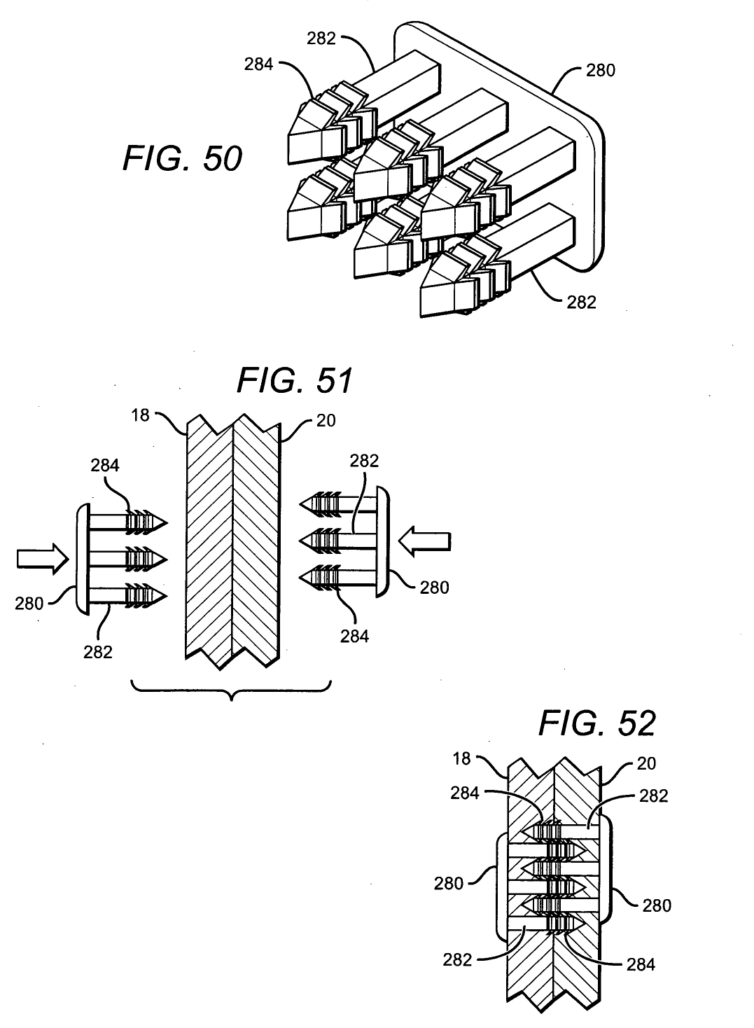

Another possibility is shown in FIGS. 50-52, in which the implant's head portion comprises a plate 280 from which at least two shafts 282 extend, each of which is pointed and includes at least one barb 284. The insertion and interlocking methods discussed above in relation to FIGS. 47-49 are applicable for this embodiment as well, as illustrated in FIGS. 51-52; note that a single implant of this sort could also be employed. Plate 280 may includes gaps (not shown) which reduce the amount of plate material pressed against the tissues being held in comparison with an equally-sized solid plate.

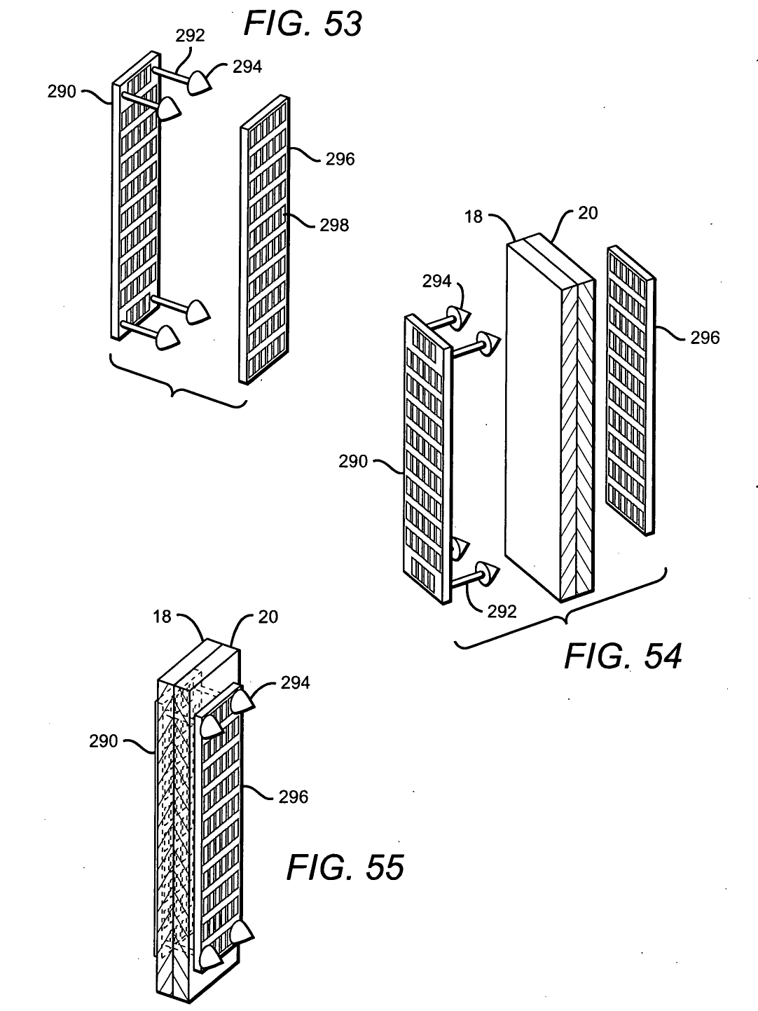

Another possible two-piece embodiment is shown in FIGS. 53-55. The first piece includes a head portion which comprises a plate 290 from which at least two shafts 292 extend, each of which is pointed and includes at least one barb 294. The second piece comprises a second plate 296 which includes gaps 298 arranged to capture the barbs 294 of plate 290 when they are pushed into gaps 298. When plates 290 and 296 are positioned on opposite sides of the tissues to be held (18, 20) and the barbs of plate 290 are captured by gaps 298, the tissues are held in approximation between the plates.

Plate 290 may be a solid plate with no gaps, or may include gaps to reduce the amount of plate material pressed against the tissues. Plate 290 may be, for example, a mesh made from a hard and/or woven material.

Though a specific configuration is shown in FIGS. 53-55, one or both of plates 290 and 296 can include gaps, one or both plates may include barbs, and there may be more or less than the four connection points shown. Many of the previously described methods of introducing barbs through tissues 18, 20 could be used, and the implant could be used for one layer of tissue or more. The mesh can be as simple as a long thin plate, or some other cross-section connected to one barb, or between a plurality of barbs.

A ‘corkscrew’ shaped implant is shown in FIGS. 56-57. Here, a biocompatible material is formed into a corkscrew shape 300, such that it can be twisted into the tissues to be held (18,20) and thereby holds the tissues in approximation between the coils.

It should be noted that all of the implant embodiments described herein can be made flexible to aid in delivery to the tissue surfaces from an angle different than perpendicular.

The embodiments of the invention described herein are exemplary and numerous modifications, variations and rearrangements can be readily envisioned to achieve substantially equivalent results, all of which are intended to be embraced within the spirit and scope of the invention as defined in the appended claims.

We claim:

1. An implant for holding two or more tissues in approximation, comprising:
a head portion; and
a first shaft which extends from said head portion, at least a portion of said implant being deformable;
such that, when said first shaft is inserted through said tissues to be held and said deformable portion is deformed, said tissues are held in approximation by said implant.

2. The implant of claim 1, wherein the end of said first shaft opposite said head portion is pointed.

3. The implant of claim 2, wherein said point is deformable, said implant arranged such that, when said point is deformed, said tissues are held in approximation between said head portion and said deformed point.

4. The implant of claim 1, further comprising a deforming die arranged to deform said deformable portion when brought into contact with said deformable portion.

5. The implant of claim 4, wherein said die is arranged to deform said deformable portion using heat or mechanical vibration.

6. The implant of claim 1, wherein said head portion comprises first and second ends, said first shaft extending from the first end of said head portion, said implant further comprising a second shaft which extends from the second end of said head portion, the ends of said first and second shafts opposite said head portion being pointed and deformable, said first and second shafts and said head portion forming a staple;
such that, when said first and second shafts are inserted through said tissues to be held and said deformable points are deformed, said tissues are held between said head portion and said deformed points.

7. The implant of claim 6, said implant further comprising one or more additional shafts which extend from said head portion, the ends of said additional shafts opposite said head portion being pointed and deformable, said shafts and said head portion forming a multi-legged staple;
such that, when said shafts are inserted through said tissues to be held and said deformable points are deformed, said tissues are held between said head portion and said deformed points.

8. The implant of claim 1, wherein said first shaft comprises one or more molly bolt-like collapsible legs, such that, when said first shaft is inserted through said tissues to be held and said collapsible legs are collapsed, said tissues are held between said head portion and said collapsed legs.

9. The implant of claim 1, wherein the end of said shaft opposite said head portion contains one or more slits extending from the tip of said shaft towards said head portion, the portions of said shaft between said slits being deformable.

10. The implant of claim 9, wherein the ends of said portions of said shaft between said slits are pointed.

11. The implant of claim 9, further comprising a deforming die arranged to split said shaft along said slits when brought into contact with said shaft's deformable portions.

12. The implant of claim 9, wherein the portions of said shaft between said slits include relief notches.

13. The implant of claim 1, further comprising a needle into or onto which said implant can be loaded, said needle arranged such that, when loaded with said implant and made to pierce said tissues to be held, said implant's shaft is delivered through said tissues along with said needle.

14. The implant of claim 13, wherein said needle is curved and said implant is curved when loaded into or onto said needle.

15. The implant of claim 13, further comprising a ram having first and second ends, said ram arranged such that, when said needle is loaded and said ram's first end abuts against the side of said head portion opposite said shaft, force applied to said ram's second end pushes said implant's head portion towards said needle's point.

16. The implant of claim 13, wherein at least a portion of said implant comprises a super-elastic material capable of being compressed while loaded into or onto said needle and then reverting to a preformed shape when said needle is retracted;
said shaft arranged to be inserted through said tissues when said super-elastic material is compressed, said implant arranged such that said tissues are held in approximation when said shaft has been inserted through said tissues and said implant is reverted to said preformed shape.

17. The implant of claim 13, wherein at least a portion of said implant comprises a shape memory material having an associated activation means, said material capable of being compressed and then reverting to a preformed shape in response to said activation means;
said shaft arranged to be inserted through said tissues when said implant is compressed, said implant arranged such that said tissues are held in approximation when said shaft has been inserted through said tissues and said implant is reverted to said preformed shape in response to said activation means.

18. The implant of claim 13, wherein said head portion includes a slot, said needle having a pointed portion and a base portion and arranged such that, when an implant is loaded into or onto said needle, said pointed portion extends through said slot and said base portion abuts against the side of said head portion opposite said shaft.

19. The implant of claim 18, wherein the end of said shaft opposite said head portion is deformable, said implant arranged such that, when said shaft is deformed, said tissues are held in approximation between said head portion and said deformed portion.

20. The implant of claim 19, further comprising a deforming die arranged to deform said deformable portion when brought into contact with said deformable portion.

21. The implant of claim 18, wherein the end of said shaft opposite said head portion comprises a shape memory material having an associated activation means, said material capable of being compressed and then reverting to a preformed shape in response to said activation means;
said implant arranged such that said tissues are held in approximation between said head portion and the shape memory portion of said shaft when said shaft has been inserted through said tissues and said implant is reverted to said preformed shape in response to said activation means.

22. The implant of claim 18, wherein the end of said shaft opposite said head portion comprises a super-elastic material capable of being compressed and then reverting to a preformed shape when said needle is retracted;
said implant arranged such that said tissues are held in approximation between said head portion and the super-elastic portion of said shaft when said shaft has been inserted through said tissues and said implant is reverted to said preformed shape.

23. The implant of claim 13, wherein said head portion and at least a portion of said shaft comprise a shape memory material having an associated activation means, said material capable of being compressed and then reverting to a preformed shape in response to said activation means;
said implant arranged such that said tissues are held in approximation between said head portion and the shape memory portion of said shaft when said shaft has been inserted through said tissues and said implant is reverted to said preformed shape in response to said activation means.

24. The implant of claim 13, further comprising a second member which includes an opening for receiving said shaft when delivered through said tissues, such that said tissues are held in approximation between said head portion and said second member.

25. The implant of claim 24, wherein said second member is a circular disc.

26. The implant of claim 24, wherein said second member includes one or more relief slits which radiate from said opening.

27. The implant of claim 24, wherein said second member initially has no opening, said opening created in said second member by said needle's point when made to pierce said tissues to be held.

28. The implant of claim 24, wherein at least a portion of said implant comprises a super-elastic or shape memory material capable of being compressed while loaded into or onto said needle and then reverting to a preformed shape when said needle is retracted;
wherein said implant's preformed shape is a T-shape with a crossbar affixed and perpendicular to one end of said shaft;
said shaft arranged to be inserted through said tissues when said implant is compressed and to revert to said T-shape when said shaft has passed completely through said tissues and said needle is retracted;
such that said tissues are held in approximation between said crossbar and said second member when said implant is reverted to said preformed shape and said second member has received said shaft.

29. The implant of claim 24, wherein at least a portion of said implant comprises a super-elastic or shape memory material capable of being compressed while loaded into or onto said needle and then reverting to a preformed shape when said needle is retracted;
wherein said implant's preformed shape is an umbrella-shape with its head portion comprising a compressible umbrella-shaped portion affixed to one end of said shaft;
said shaft arranged to be inserted through said tissues when said implant is compressed and to revert to said umbrella-shape when said shaft has passed completely through said tissues and said needle is retracted;
such that said tissues are held in approximation between said head portion and said second member when said implant is reverted to said preformed shape and said second member has received said shaft.

30. The implant of claim 29, wherein said compressible umbrella-shaped head portion includes one or more slits which divide the umbrella-shaped head portion into two or more segments, each of which can be compressed along the length of said shaft.

31. The implant of claim 30, wherein said slits between said segments are generally triangular.

32. The implant of claim 13, wherein at least a portion of said implant comprises a super-elastic or shape memory material capable of being compressed while loaded into or onto said needle and then reverting to a preformed shape when said needle is retracted;
said implant further comprising one or more additional shafts which extend from said head portion, each of said additional shafts comprising:
an arm portion coupled to said head portion; and
a finger portion on the end of said shaft opposite said head portion, said implant forming a compressed clip when loaded into or onto said needle;
said implant's preformed shape being an uncompressed clip;
said shafts arranged to be inserted through said tissues when said implant is compressed and to revert to said uncompressed clip-shape when said shaft has passed completely through said tissues and said needle is retracted;
such that said tissues are held in approximation between said head portion and said fingers when said implant is reverted to said preformed shape

33. The implant of claim 32, wherein the side of said head portion opposite said arms is pointed to aid in piercing said tissues.

34. The implant of claim 32, wherein each of said arm portions includes at least one barb to aid in the fixation of said arms to said tissues.

35. The implant of claim 1, further comprising one or more additional shafts which extend from said head portion, the ends of each of said shafts opposite said head portion being pointed, said shafts and said head portion forming a multi-legged staple;
wherein at least a portion of said implant comprises a super-elastic material capable of being compressed and then reverting to a preformed shape when unconstrained;
said implant arranged such that said tissues are held in approximation when said implant is reverted to said preformed shape.

36. The implant of claim 1, further comprising one or more additional shafts which extend from said head portion, the ends of each of said shafts opposite said head portion being pointed, said shafts and said head portion forming a multi-legged staple;
wherein at least a portion of said implant comprises a shape memory material having an associated activation means, said material capable of being compressed and then reverting to a preformed shape in response to said activation means;
said implant arranged such that said tissues are held in approximation when said implant is reverted to said preformed shape in response to said activation means.

37. The implant of claim 1, wherein said first shaft extends perpendicularly from said head portion.

38. The implant of claim 1, wherein said implant comprises an absorbable or non-absorbable biocompatible plastic.

39. The implant of claim 38, wherein said plastic is super-elastic.

40. The implant of claim 1, wherein said implant comprises a biocompatible metal.

41. The implant of claim 40, wherein said metal is super-elastic.

42. The implant of claim 40, wherein said metal comprises nickel-titanium.

43. The implant of claim 40, wherein said metal comprises NITINOL.

44. The implant of claim 40, wherein said metal comprises stainless steel.

45. The implant of claim 40, wherein said metal comprises a shape memory material.

46. The implant of claim 45, wherein said shape memory material is temperature-sensitive, said material capable of being compressed and then reverting to a preformed shape when subjected to an activation temperature, said shape memory material arranged to revert to its preformed state when subjected to normal body temperatures.

47. A implant for holding two or more tissues in approximation, comprising:
a head portion; and
at least one shaft which extends from said head portion, each of said shafts including at least one barb;
such that, when said at least one shaft is inserted through said tissues to be held, said tissues are held in approximation between said head portion and said at least one barb.

48. The implant of claim 47, wherein said at least one shaft comprises at least two shafts, each of which is pointed and extends from said head portion at an angle such that, when pushed into said tissues to be held, said shafts are spread apart and said barbs are forced into said tissues.

49. The implant of claim 47, wherein said at least one shaft comprises at least two shafts, each of which is pointed, said shafts extending from said head portion and cantered towards each other such that, when pushed into said tissues to be held, said shafts are pushed together and said barbs are forced into said tissues.

50. The implant of claim 47, further comprising at least one additional implant, each of said additional implants comprising:
a head portion; and
at least one shaft which extends from said head portion, each of said shafts being pointed and including at least one barb;
at least two of said implants inserted into said tissues to be held from opposite directions.

51. The implant of claim 50, wherein said implants are arranged such that two of said implants inserted into said tissues from opposite directions can intersect and lock onto each other via their respective barbs.

52. The implant of claim 47, wherein said head portion comprises a plate from which at least two of said shafts extend, each of said shafts being pointed and including at least one barb;
such that, when said shafts are inserted through said tissues to be held, said tissues are held in approximation between said plate and said barbs.

53. The implant of claim 52, wherein said implants are arranged such that two of said implants inserted into said tissues from opposite directions can intersect and lock onto each other via their respective barbs.

54. The implant of claim 52, wherein said plate includes gaps which reduce the amount of plate material pressed against said tissues in comparison with an equally-sized solid plate.

55. The implant of claim 52, wherein said head portion comprises a first plate from which at least two of said shafts extend, each of said shafts being pointed and including at least one barb;
further comprising a second plate, said second plate including gaps arranged to capture the barbs of said first plate when the barbs of said first plate are pushed into said gaps,
such that, when said first and second plates are positioned on opposite sides of said tissues to be held and the barbs of said first plate are captured by the gaps of said second plate, said tissues are held in approximation between said first and second plates.

56. A implant for holding two or more tissues in approximation, comprising:
a biocompatible material formed into a corkscrew shape such that it can be twisted into said tissues to be held,
such that, when twisted into said tissues to be held, said tissues are held in approximation between the coils of said corkscrew-shaped material.
